(19) United States
(12) Patent Application Publication
(10) Pub. No.: US 2024/0376167 A1
(43) Pub. Date: Nov. 14, 2024

(54) ANIMAL FREE ALPHA S1 AND ALPHA S2 CASEIN-FUSION MILK PROTEIN, PROCESS FOR PREPARING THE SAME

(71) Applicant: HENI INNOVATION PRIVATE LIMITED, Surat (IN)

(72) Inventor: PARINI KAPADIA, Gujarat (IN)

(73) Assignee: HENI INNOVATION PRIVATE LIMITED, Surat (IN)

(21) Appl. No.: 18/638,342

(22) Filed: Apr. 17, 2024

(30) Foreign Application Priority Data
Apr. 17, 2023 (IN) 202321027946

(51) Int. Cl. C07K 14/47; A23C 11/08; C07K 1/22; C12N 1/20; C12N 15/70; C12N 15/81; C12Q 1/24; C12Q 1/686; C12R 1/19; G16B 20/00

(52) U.S. Cl. CPC C07K 14/4732; A23C 11/08; C07K 1/22; C12N 1/205; C12N 15/70; C12N 15/81; C12Q 1/24; C12Q 1/686; G16B 20/00; C07K 2319/21; C12R 2001/19

(57) ABSTRACT

The present invention broadly relates to the field of biotechnology. More particularly, the present invention relates to animal-free fusion milk proteins and a process for the production of the same in recombinant host cells. Also, the present invention provides vectors, and expression cassettes for the expression of animal-free milk proteins in host cells.

### CROSS-REFERENCE TO RELATED APPLICATIONS

This application is a Paris Convention U.S. national phase application which claims priority to and the benefit of Indian Patent Application No. 202321027946, filed Apr. 17, 2023, the content of which is incorporated herein by reference in its entirety.

### SEQUENCE LISTING

The instant application contains a Sequence Listing which has been submitted electronically in XML file format and is hereby incorporated by reference in its entirety. Said XML copy, created on Feb. 29, 2024, is named Sequence listing.xml and is 9,565 bytes in size.

### FIELD OF THE INVENTION

The present invention broadly relates to the field of biotechnology. More particularly, the present invention relates to animal-free fusion milk proteins and a process for the production of the same in recombinant host cells. Also, the present invention provides vectors, and expression cassettes for the expression of animal-free milk proteins in host cells.

### BACKGROUND ART

Globally, more than 7.5 billion people around the world consume milk and milk products. Demand for cow milk and dairy products is expected to keep increasing due to increased reliance on these products in developing countries as well as growth in the human population, which is expected to exceed 9 billion people by 2050.

Relying on animal agriculture to meet the growing demand for food is not a sustainable solution. According to the Food & Agriculture Organization of the United Nations, animal agriculture is responsible for 18% of all greenhouse gases, more than the entire transportation sector combined. Dairy cows alone account for 3% of this total. Various studies have identified that the dairy industry is one of the most common contributors to the greenhouse gas emissions. More particularly, it is known that dairy production is the contributor for emission of greenhouse gases such as methane, nitrous oxide and carbon dioxide. As a result, by avoiding meat and dairy products in our day-to-day diet, it can substantially reduce the impact on the planet. Further people with lactose intolerance are unable to fully digest the sugar (lactose) in natural cow milk. Lactose intolerance is nothing but the inability to break down the lactose and the reason for the same is due to humans losing ability to produce enough lactase enzyme as they age to digest and break down the lactose. The natural cow milk also contains antibiotics, hormone, cholesterol and saturated fat which may not be always good for humans.

In addition to impacting the environment, animal agriculture poses a serious risk to human health. A startling 80% of antibiotics used in the United States go towards treating animals, resulting in the development of antibiotic resistant microorganisms also known as superbugs. For years, food companies and farmers have administered antibiotics not only to sick animals, but also to healthy animals, to prevent illness. In September 2016, the United Nations announced the use of antibiotics in the food system as a crisis on par with Ebola and HIV.

It is estimated that cow milk accounts for 83% of global milk production. Accordingly, there is an urgent need for to provide bovine milk and/or essential high-quality proteins from bovine milk in a more sustainable and humane manner, instead of solely relying on animal farming.

In recent times, cellular agriculture has gained tremendous interest and has a variety of applications to address public health, environmental and animal welfare, and food security. The cellular agriculture is defined as the production of agricultural products from cell cultures rather than from whole plants or animals. The cellular agriculture is a promising technology in the food science, environmental science, nutrition, and dietetics research areas.

In the article published by Harvard Library, 90 Reasons to Consider Cellular Agriculture, disclosed that due to contamination-free production methodologies of cellular agriculture, foods can stay safer for a greater duration, ultimately granting a longer shelf life. The cellular agriculture can supply the growing demand for animal products, feeding the increasing world population of almost 10 billion by 2050. Moreover, the cellular agriculture dairy production processes may require 97% less land and 99.6% less water, may produce 65% less greenhouse gas emissions. There would be less waste of dairy products in a cellular agriculture system, since there is greater product control.

Accordingly, there is a need for the development of a synthetic process for the preparation of milk proteins which substantially reduces the greenhouse emissions that is observed in the conventional methods. The inventors of the present invention have by using genetic engineering developed animal-free milk proteins and an efficient process for preparation of the same. The dairy products containing such animal-free fusion proteins will be safe, delicious and identical to the naturally available bovine milk and milk proteins.

### SUMMARY OF THE INVENTION

The present invention relates to animal-free fusion milk proteins and a process for the production of fusion milk proteins in recombinant host cells. Further, the present invention also provides a recombinant vector system for the expression of target proteins in host cells. This vector system comprises a recombinant polynucleotide sequence encoding polypeptide sequences for the target fusion milk protein.

Lastly, the present invention also provides method for the purification and isolation of the target fusion milk protein.

### Object of the Present Invention

An object of the present invention provides an animal-free fusion protein encoding milk proteins that mimic the naturally occurring milk proteins from Bos indicus.

An object of the present invention provides a method for the production of animal-free milk proteins in recombinant host cells.

An object of the present invention provides a recombinant vector comprising the polynucleotide sequence encoding the fusion milk protein.

An object of the present invention provides a recombinant polynucleotide sequence is a sequence encoding the milk fusion protein.

An object of the present invention provides a composition comprising the fusion protein along with pharmaceutically or nutraceutically acceptable carriers or vehicles.

An object of the present invention provides a nucleic acid sequence encoding the recombinant fusion milk protein.

An object of the present invention provides a recombinant host cell comprising the nucleic acid sequence encoding the recombinant fusion milk protein.

An object of the present invention provides a food composition comprising the fusion milk protein of the present invention.

### BRIEF DESCRIPTION OF THE ACCOMPANYING FIGURES

The accompanying drawings illustrate some of the embodiments of the present disclosure and, together with the descriptions, serve to explain the disclosure. These drawings have been provided by way of illustration and not by way of limitation. The components in the drawings are not necessarily drawn to scale, emphasis instead being placed upon clearly illustrating the principles of the aspects of the embodiments.

FIG. 1: In-house culture of competent E. coli DH110β

FIG. 2: LB agar medium containing antibiotic with transformed E. coli DH10β cells and control LB agar plates.

FIG. 3: Microcentrifuge or falcon tube containing DNA+competent cells E. coli DH10β

FIG. 4: Agarose gel electrophoresis of the recombinant vector containing pD912 AS1+AS2 casein.

FIG. 5: Agarose gel electrophoresis of the circular and linear DNA containing pD912 AS1+AS2 casein.

FIG. 6: αS1+αS2 Fusion protein patched on YPDA+100 μg/mL Zeocin

FIG. 7: αS1+αS2 Fusion protein patched on YPDA+200 & 500 μg/mL Zeocin

FIG. 8: αS1+αS2 Fusion protein YPD broth+200 μg/mL Zeocin

FIG. 9: αS1+αS2 Fusion protein BMGY & BMMY medium

FIG. 10 (a, b, c): Screening & Protein overexpression studies—AS1 & AS2 Fusion protein (sample 16) at 96 hours, 144 hours and 120 hours.

FIG. 11—Dot blot Assay—AS1 & AS2 Fusion protein (sample 16)

FIG. 12—HPLC chromatogram of Standard Alpha casein (SIGMA)

FIG. 13—HPLC chromatogram of Recombinant fusion protein

### DETAILED DESCRIPTION FOR SEQUENCE LISTING

SEQ ID No. 1—amino acid sequence encoding Alpha S1+Alpha S2 casein Fusion protein.

SEQ ID No. 2—polynucleotide sequence encoding Alpha S1+Alpha S2 casein Fusion protein.

SEQ ID No. 3—amino acid sequence encoding Alpha S1 Casein protein.

SEQ ID No. 4—polynucleotide sequence encoding Alpha S1 Casein protein.

SEQ ID No. 5—amino acid sequence encoding Alpha S2 Casein protein.

SEQ ID No. 6—polynucleotide sequence encoding Alpha S2 Casein protein.

### DETAILED DESCRIPTION OF THE INVENTION

At the very outset of the detailed description, it may be understood that the ensuing description only illustrates a particular form of this invention. However, such a particular form is only exemplary embodiment, and without intending to imply any limitation on the scope of this invention. Accordingly, the description is to be understood as an exemplary embodiment and teaching of invention and not intended to be taken restrictively.

Unless defined otherwise, technical and scientific terms used herein have the same meaning as commonly understood by one of ordinary skill in the art to which this invention belongs. Further, unless otherwise required by context, singular terms shall include pluralities and plural terms shall include the singular.

### Definitions

While the following terms are believed to be well understood by one of ordinary skill in the art, the following definitions are set forth to facilitate explanation of the presently disclosed subject matter.

The term “milk protein” means a protein that is found in a mammal-produced milk or a protein having a sequence that is at least 80% identical (e.g., at least 85%, at least 90%, at least 95%, at least 96%, at least 97%, at least 98%, or at least 99% identical) to the sequence of a protein that is found in a mammal-produced milk. Non-limiting examples of milk proteins include: Beta-casein, Kappa-casein, Alpha-S1-casein, Alpha-S2-casein, alpha-lactalbumin, Beta-lactoglobulin, Lactoferrin, Transferrin, and Serum albumin. Additional milk proteins are known in the art.

The term “mammal-produced milk proteins” is known in the art and means a milk protein produced by a mammal.

The term “animal-free milk proteins” is known in the art and means any milk proteins that are not produced by a mammal.

As used herein, the term “fusion protein” refers to a protein comprising at least two constituent proteins (or fragments or variants thereof) that are encoded by separate genes, and that have been joined so that they are transcribed and translated as a single polypeptide. In some embodiments, a fusion protein may be separated into its constituent proteins, for example by cleavage with a protease.

The term “linker” as used herein means an oligonucleotide sequence that links two polynucleotide sequences encoding two heterologous or homologous proteins.

The term “genetically engineered” as used herein relates to an organism that includes exogeneous, non-native nucleic acid sequences either maintained episomally in an expression vector or integrated into the genome of the host organism.

The term “recombinant” refers to nucleic acids or proteins formed by laboratory methods of genetic recombination (e.g., molecular cloning) to bring together genetic material from multiple sources, creating sequences that would not otherwise be found in the genome. A recombinant fusion protein is a protein created by combining sequences encoding two or more constituent proteins, such that they are expressed as a single polypeptide. Recombinant fusion proteins may be expressed in vivo in various types of host cells, including plant cells, bacterial cells, fungal cells, mammalian cells, etc. Recombinant fusion proteins may also be generated in vitro.

The term “recombinant” is an art known term. When referring to a nucleic acid (e.g., a gene), the term “recombinant” can be used, e.g., to describe a nucleic acid that has been removed from its naturally occurring environment, a nucleic acid that is not associated with all or a portion of a nucleic acid abutting or proximal to the nucleic acid when it is found in nature, a nucleic acid that is operatively linked to a nucleic acid which it is not linked to in nature, or a nucleic acid that does not occur in nature. The term “recombinant” can be used, e.g., to describe cloned DNA isolates, or a nucleic acid including a chemically synthesized nucleotide analog. When “recombinant” is used to describe a protein, it can refer to, e.g., a protein that is produced in a cell of a different species or type, as compared to the species or type of cell that produces the protein in nature.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion, or a point mutation introduced artificially, e.g., by human intervention. A “recombinant nucleic acid” also includes a nucleic acid integrated into a host cell chromosome at a heterologous site and a nucleic acid construct present as an episome.

The term “vector” as used herein is intended to refer to a nucleic acid molecule capable of transporting another nucleic acid to which it has been linked.

One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell and are thereby replicated along with the host genome. Moreover, certain preferred vectors are capable of directing the expression of genes to which they are operatively linked. Such vectors are referred to herein as “recombinant expression vectors” (or simply “expression vectors”).

Promoters useful for expressing the recombinant genes described herein include both constitutive and inducible/repressible promoters. Examples of inducible/repressible promoters include Methanol inducible promotor (AOX1). Where multiple recombinant genes are expressed in an engineered yeast, the different genes can be controlled by different promoters or by identical promoters in separate operons, or the expression of two or more genes may be controlled by a single promoter as part of an operon.

The term “operably linked” expression control sequences refer to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and as used herein refer to polynucleotide sequences which are necessary to affect the expression of coding sequences to which they are operably linked. Expression control sequences are sequences which control the transcription, post-transcriptional events, and translation of nucleic acid sequences. Expression control sequences include appropriate transcription initiation, termination, promoter and enhancer sequences; efficient RNA processing signals, such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “transfect”, “transfection”, “transfecting,” and the like refer to the introduction of a heterologous nucleic acid into eukaryote, prokaryotic or yeast or fungal cells.

“Transformation” refers to a process by which a nucleic acid is introduced into a cell, either transiently or stably. Transformation may rely on any known method for the insertion of nucleic acid sequences into a prokaryotic or eukaryotic host cell, including Agrobacterium-mediated transformation protocols, viral infection, whiskers, electroporation, heat shock, lipofection, polyethylene glycol treatment, micro-injection, and particle bombardment.

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism. Host cells can be any organism selected from the group consisting of bacteria, yeast and filamentous fungi.

The term “yeast and filamentous fungi” include, but are not limited to any Kluyveromyces sp., such as Kluyveromyces lactis, Kluyveromyces marxianus, Saccharomyces sp., such as Saccharomyces cerevisiae, Pichia sp., such as Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Hansenula polymorpha, Candida albicans, any Aspergillus sp., such as Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens, and Neurospora crassa.

As used herein, the term “predominantly” or variations thereof will be understood to mean, for instance, a) in the context of fats the amount of a particular fatty acid composition relative to the total amount of fatty acid composition; b) in the context of protein the amount of a particular protein composition relative to the total amount of protein composition.

The term “about,” “approximately,” or “similar to” means within an acceptable error range for the particular value as determined by one of ordinary skill in the art, which can depend in part on how the value is measured or determined, or on the limitations of the measurement system. It should be understood that all ranges and quantities described below are approximations and are not intended to limit the invention. Where ranges and numbers are used these can be approximate to include statistical ranges or measurement errors or variation. In some embodiments, for instance, measurements could be plus or minus 10%.

The phrase “essentially free of” is used to indicate the indicated component, if present, is present in an amount that does not contribute, or contributes only in a de minimus fashion, to the properties of the composition. In various embodiments, where a composition is essentially free of a particular component, the component is present in less than a functional amount. In various embodiments, the component may be present in trace amounts. Particular limits will vary depending on the nature of the component, but may be, for example, selected from less than 10% by weight, less than 9% by weight, less than 8% by weight, less than 7% by weight, less than 6% by weight, less than 5% by weight, less than 4% by weight, less than 3% by weight, less than 2% by weight, less than 1% by weight, or less than 0.5% by weight.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context.

### DETAILED DESCRIPTION

Bovine milk and dairy products have long traditions in human nutrition. Bovine milk contains the nutrients needed for growth and development of the calf, and is a resource of proteins, lipids, vitamins, amino acids and minerals. Bovine milk also contains growth factors, immunoglobulins, hormones, cytokines, peptides, nucleotides, enzymes, polyamines and other bioactive peptides. Milk proteins are important components for colour, taste and texture of the milk. Milk proteins play a very important role for the production of different kind of dairy products. Milk is consumed as a solo beverage and is also consumed in different forms like cheese, paneer, yogurt, coffee, flavoured drinks etc. Industry also has Ghee as the most consumed value-added dairy product following non-fat milk and butter.

Milk contains two types of protein Casein and Whey. Milk protein composition varies depending on the species (example cow, goat, sheep), breed (example Holstein, Jersey), the animal's feed, and the stage of lactation.

### Casein

Casein proteins represents a family of proteins that is present in mammal-produced milk and is capable of self-assembling with other proteins in the family to form micelles and/or precipitate out of an aqueous solution at an acidic pH. Non-limiting examples of casein proteins include Beta-casein, Kappa-casein, Alpha-S1-casein, and Alpha-S2-casein. Non-limiting examples of sequences for casein protein from different mammals are provided herein. Additional sequences for other mammalian caseins are known in the art. In the context of the invention, a “casein protein” refers to a protein at least 30%, at least 40%, at least 50%, at least 60%, at least 70%, at least 80%, at least 90%, at least 95% or completely identical to a natural mammalian casein protein. Preferred casein proteins are bovine casein proteins.

### Alpha-S1-Casein and Alpha-S2-Casein

The Alpha casein fractions also play an important role in casein micelle formation as their presence has a positive effect. Alpha-casein is the largest fraction and includes those phosphoproteins precipitated at low calcium concentrations. The Alpha S1-casein family makes up 40% of this fraction and contains 214 amino acid residues, of which 8.4% are prolyl residues evenly distributed throughout the polypeptide chain. The Alpha S2-casein accounts for 10% of this fraction and is composed of 222 amino acid residues. It is the most hydrophilic of the caseins, with 10 to 13 phosphoryl residues.

### Fusion Protein

In an aspect, the present invention provides fusion proteins encoding the milk proteins. A fusion protein is a protein comprising at least two constituent proteins (or fragments or variants thereof) that are encoded by separate genes, and that have been joined so that they are transcribed and translated as a single polypeptide. In another aspects, the two proteins are linked together by a linker peptide sequence. The recombinant polypeptides are encoded by the host cells which produce this fusion protein. The fusion proteins are then isolated and purified by methods known in the art. In another aspect, the fusion protein of the present invention comprises of a small protein or peptide (tag) in addition to the protein of interest to aid purification of recombinant proteins. Fusion tags can improve protein expression, stability, resistance to proteolytic degradation and solubility. Three of the most important uses of fusion proteins are: as aids in the purification of cloned genes, as reporters of expression level, and as histochemical tags to enable visualization of the location of proteins in a cell, tissue, or organism. DNA sequences encoding targeted genes with a C-terminal HIS-tag/will be codon-optimized for expression in host strains.

### Nucleic Acids and Vectors

In an aspect, the present invention also provides nucleic acids (e.g., vectors) that include: a promoter (e.g., a yeast, bacterial, or a mammalian promoter); a sequence encoding a signal sequence; a sequence encoding a milk protein (e.g., any of the exemplary sequences described herein); and a yeast termination sequence, where the promoter is operably linked to the signal sequence, the signal sequence is operably linked to the sequence encoding the milk protein, and the terminal sequence is operably linked to the sequence encoding the milk protein. In some examples of these nucleic acids, the promoter is a constitutive promoter or an inducible promoter. Additional promoters that can be used in these nucleic acids are known in the art.

The signal sequence in any of the vectors described herein can be a signal sequence from the encoded milk protein or a different milk protein or is a signal sequence from a yeast mating factor (e.g., any alpha mating factor). In some aspects, the encoded milk protein is selected from the group of: Beta-casein (e.g., any of the Beta-casein proteins described herein), Kappa-casein (e.g., any of the Kappa-casein proteins described herein), Alpha-S1-casein (e.g., any of the Alpha-S1-casein proteins described herein), Alpha-S2-casein (e.g., any of the Alpha-S2-casein proteins described herein), Alpha-lactalbumin (e.g., any of the Alpha-lactalbumin proteins described herein), Beta-lactoglobulin (e.g., any of the Beta-lactoglobulin proteins described herein), Lactoferrin (e.g., any of the lactoferrin proteins described herein), or Transferrin (e.g., any of the transferrin proteins described herein). Additional signal sequences that can be used in the present vectors are known in the art.

Any of the nucleic acids described herein can further include a bacterial origin of replication. Any of the nucleic acids described herein can further include a selection marker (e.g., an antibiotic resistance gene). The sequences of bacterial origin of replication are known in the art. non-limiting examples of antibiotic resistance genes are described herein. Additional examples of resistance genes are known in the art. non-limiting examples of termination sequences are described herein. Additional examples of termination sequences are known in the art.

In some aspects, the nucleic acids provided herein further include: an additional promoter sequence (e.g., any of the exemplary promoters described herein); an additional sequence encoding a signal sequence (e.g., any of the exemplary signal sequences described herein); a sequence encoding an additional milk protein (e.g., any of the exemplary sequences encoding a milk protein described herein); and an additional yeast termination sequence (e.g. any of the exemplary yeast termination sequences described herein), where the additional promoter sequence is operably linked to the additional sequence encoding a signal sequence, the sequence encoding the signal sequence is operably linked to the sequence encoding the additional milk protein, and the sequence encoding the additional milk protein is operably linked to the additional yeast terminal sequence. The additional milk protein can be, e.g., Beta-casein (e.g., any of the Beta-casein proteins described herein), Kappa-casein (e.g., any of the Kappa-casein proteins described herein), Alpha-S1-casein (e.g., any of the Alpha-S1-casein proteins described herein), Alpha-S2-casein (e.g., any of the Alpha-S2-casein proteins described herein), Alpha-lactalbumin (e.g., any of the Alpha-lactalbumin proteins described herein), Beta-lactoglobulin (e.g., any of the Beta-lactoglobulin proteins described herein), Lactoferrin (e.g., any of the lactoferrin proteins described herein), or Transferrin (e.g., any of the transferrin proteins described herein). In some embodiments, the nucleic acid includes a sequence encoding a Beta-casein and a sequence encoding a Kappa-casein. The promoter and the additional promoter can be the same or different. The yeast termination sequence and the additional yeast terminal sequence can be the same or different. The signal sequence and the additional signal sequence can be the same or different.

In an aspect, the present invention also encompasses a recombinant vector system containing the isolated DNA sequence encoding either casein or whey polypeptide and host cells comprising the vector. The vector may further comprise an isolated DNA sequence comprising a nucleotide sequence encoding a casein, wherein the nucleotide sequence is operably linked to a promoter, a nucleotide sequence encoding an alpha mating factor, or a variant thereof, a nucleotide sequence encoding a bacterial resistance marker and a transcription terminator. Alternatively, the vector may further comprise an isolated DNA sequence comprising a nucleotide sequence encoding whey protein, wherein the nucleotide sequence is operably linked to a promoter, a nucleotide sequence encoding an alpha mating factor, or a variant thereof, a nucleotide sequence encoding a bacterial resistance marker and a transcription terminator. One or more of suitable promoters are utilized for expression of the genes encoding casein or whey proteins may be any promoter which is functional in the host cell and is able to elicit expression of the product encoded by the gene.

Also provided herein are examples of expression cassettes for the expression of casein or whey proteins in non-mammalian systems, such as plants and microorganisms, to produce recombinant casein proteins. The expression cassette may comprise, for example, a promoter, a 5′ untranslated region (UTR), a sequence encoding one or more casein proteins, and a terminator. The expression cassette may further comprise a selectable marker and retention signal.

In some aspect, a nucleic acid comprises a sequence encoding a fusion protein. In some embodiments, a nucleic acid comprises a sequence encoding a fusion protein, which is operably linked to a promoter. In another aspect, a nucleic acid comprises, in order from 5′ to 3′, a promoter, a 5′ untranslated region (UTR), a sequence encoding a 1^{st} milk protein—a sequence encoding a linker peptide—a sequence encoding the 2^{nd} milk protein, and a terminator.

Introducing Nucleic Acids into a Cell

Methods of introducing nucleic acids (e.g., any of the nucleic acids described herein) into a cell to generate a host cell are well-known in the art. Non-limiting examples of techniques that can be used to introduce a nucleic acid into a cell include: calcium phosphate transfection, dendrimer transfection, liposome transfection (e.g., cationic liposome transfection), cationic polymer transfection, electroporation, cell squeezing, sonoporation, optical transfection, protoplast fusion, impalefection, hyrodynamic delivery, gene gun, magnetofection, and viral transduction.

One skilled in the art would be able to select one or more suitable techniques for introducing the nucleic acids into a cell based on the knowledge in the art that certain techniques for introducing a nucleic acid into a cell work better for different types of host cells. Exemplary methods for introducing a nucleic acid into a yeast cell are described in Kawai et al., Bioeng. Bugs 1:395-403, 2010.

### Host Cells

In an aspect, the present invention provides host cells including any of the nucleic acids (e.g., vectors) described herein. In another aspect, the nucleic acid described herein is stably integrated within the genome (e.g., a chromosome) of the host cell. In other aspects, the nucleic acid described herein is not stably integrated within the genome of the host cell.

In some aspects of the present invention, the host cell is a yeast strain or a bacterial strain. an organism selected from the group consisting of bacteria, yeast and filamentous fungi. In a preferred aspect, the host cell can be, e.g., a yeast strain selected from the group of:
a Kluyveromyces sp., Pichia sp., Saccharomyces sp., Tetrahymena sp., Yarrowia sp., Hansen ula sp., Blastobotrys sp., Candida sp., Zygosaccharomyces sp., Trichoderma sp., and Debaryomyces sp. Additional non-limiting examples of yeast strains that can be used as the host cell are Kluyveromyces lactis, Kluyveromyces marxianus, Saccharomyces cerevisiae, Trichoderma reseei and Pichia pastoris. Additional species of yeast strains that can be used as host cells are known in the art.

In another aspect, the host cell can be a protozoa, such as, e.g., Tetrahymena thermophile, T. hegewischi, T. hyperangularis, T. malaccensis, T. pigmentosa, T pyriformis, and T. vorax.

In another aspect, the present invention provides a method for isolating the animal-free milk fusion proteins components by recombinantly expressing them in any of the host cells provided herein.

### EMBODIMENTS OF THE PRESENT INVENTION

An important embodiment of the present invention relates to a fusion protein encoded by a recombinant polypeptide sequence having an amino acid sequence having 90% similarity to SEQ ID No. 1, wherein the said fusion protein is an animal free milk protein that mimic the naturally occurring milk proteins.

Another important embodiment of the present invention relates to the recombinant polypeptide sequence, SEQ ID No. 1, comprising an amino acid sequence having 90% similarity to SEQ ID No. 3 and an amino acid sequence having 90% similarity to SEQ ID No. 5.

Another important embodiment of the present invention relates to the fusion protein bovine milk proteins are selected from any bovine milk proteins selected from the group comprising of Beta-casein, Kappa-casein, Alpha-S1-casein, Alpha-S2-casein, Alpha-lactalbumin, Beta-lactoglobulin, Lactoferrin, and/or Transferrin.

A preferred embodiment of the present invention provides that the bovine milk proteins are Alpha-S1-casein and Alpha-S2-casein.

Another important embodiment provides that the amino acid sequences encoding Alpha-S1-casein and Alpha-S2-casein have been obtained from Gir cattle (Bos indicus).

Yet another important embodiment of the present invention provides a process for the production of animal-free milk proteins in recombinant host cells, the process comprising the steps of:
i. identifying high quality milk producing Gir cattle (Bos indicus) by using various bioinformatics techniques and analysis;
ii. performing genetic analysis of identified Gir cattle from step (i) to identify the specific genetic segment which is responsible for production of desired milk proteins;
iii. performing in-silico data analysis and codon optimization of gene sequences for the requisite milk proteins to obtain the gene sequences;
iv. Preparing the recombinant vector comprising the recombinant polynucleotide sequence having SEQ ID No. 2 obtained from step (iii);
v. transforming the recombinant vector obtained in step (iv) into the host cell; and
vi. culturing the recombinant host cells to express the animal free milk protein.

Another important embodiment of the present invention provides the process for the production of animal-free milk proteins in recombinant host cells, wherein the step (iv) of the method comprises the steps of:
a) preparing DNA fragment(s) from a linearized plasmid vector by restriction enzyme digestion or by PCR techniques;
b) amplifying DNA fragment(s) obtained in step (a) from the insert by PCR techniques while ensuring that the sequence at the end of the fragment is homologous with that of the plasmid;
c) mixing amplified DNA obtained in step (b) with competent cells in a microcentrifuge or falcon tube, followed by gentle mixing by flicking the bottom of the tube a few time;
d) incubating the competent cell/DNA mixture obtained in step (c) on ice;
e) performing heat shock transformation by placing the microcentrifuge tube into a water bath followed by placing the tubes back on ice for to obtained the transformed E. coli cells;
f) adding Luria Broth (LB) or (SOC) media to the tube containing the transformed E. coli cells and incubating the tubes in a shaking incubator;
g) plating the grown transformed cells onto a low salt LB agar plate containing the antibiotic and incubating the plates overnight;
h) selecting the positive transformants by identifying the plasmid vector marker;
i) recovering the constructed vector from the transformed host cell into a second host cell to amplify and identify the structure of the plasmid with restriction enzyme digestion and DNA sequencing.

Another embodiment of the present invention provides the process for the production of animal-free milk proteins in recombinant host cells, wherein the step (v) of the method comprises the steps of:
a) digesting the recombinant vector obtained in step (iv) of the process, with the restriction enzyme at an appropriate temperature and time to obtain the linear vector;
b) boiling the single-stranded linear vector followed by immediate chilling in ice water;
c) culturing host cells in YPD media with shaking followed by harvesting the cells and washing with sterile water;
d) centrifuging the solution obtained in step (c) at room temperature and the water from the solution was decanted followed with suspending the cells in LiCl;
e) transferring the cell suspension obtained in step (d) to a microcentrifuge tube and centrifuging the tube at maximum speed followed by removing the LiCl with a pipette;
f) resuspending the cells in LiCl followed by dispensing the cell suspension into a microcentrifuge tube for carrying out transformation studies;
g) adding to the transformation tubes PEG, LiCl, single-stranded plasmid DNA obtained in step (f) followed by incubating the tube without shaking;
h) subjecting the tubes to heat shock by placing them in a water bath;
i) adding YPD to the tubes obtained in step (h) and kept for overnight for culturing the transformed cells followed by plating the cells on YPD plates and incubating the plates to obtain the transformed cells.

Another embodiment of the present invention provides the process for the production of animal-free milk proteins in recombinant host cells, wherein the step (vi) of the method comprises the steps of:
a) screening of positive transformant host cells obtained in step (v) of the method using PCR and nucleotide sequencing;
b) isolating and screening multiple clones from each transformed host cells for expression of secreted target proteins;
c) regrowing the best clones isolated in step (b) in shake flasks to confirm expression;
d) extracting target fusion protein from the transformed host cells by performing affinity chromatography to isolate the target fusion proteins;
e) performing downstream processing to purify the target fusion milk proteins.

Another embodiment of the present invention provides that the host cell is selected from bacterial, yeast or fungal cells.

A preferred embodiment of the present invention provides that the host cell is a yeast cell.

Another embodiment of the present invention provides that the downstream purification process can be carried out by any method including centrifugation, column chromatography and/or membrane filtration.

Yet another important embodiment of the present invention provides a composition comprising the fusion protein along with pharmaceutically or nutraceutically acceptable carriers or vehicles.

Yet another important embodiment of the present invention provides a nucleic acid sequence encoding the recombinant fusion protein encoded by the recombinant polynucleotide sequence having 90% similarity to SEQ ID No. 2

Yet another important embodiment of the present invention provides that the recombinant polynucleotide sequence, SEQ ID No. 2, comprises polynucleotide sequences having 90% similarity to SEQ ID No. 4 and SEQ ID No. 6.

Yet another important embodiment of the present invention provides a recombinant vector comprising the nucleic acid sequence wherein the nucleic acid sequence encoding the recombinant fusion protein encoded by the recombinant polynucleotide sequence having 90% similarity to SEQ ID No. 2.

Another embodiment of the present invention provides that the recombinant vector comprising the polynucleotide sequence operably linked to a promoter, a nucleotide sequence encoding a ‘HIS’ tag, a nucleotide sequence encoding a bacterial resistance marker and a transcription terminator.

Yet another important embodiment of the present invention provides a recombinant host cell comprising the nucleic acid sequence encoding the recombinant fusion protein having 90% similarity to SEQ ID No. 1.

A preferred embodiment of the present invention provides that the host cell is selected from bacterial, plant, yeast or fungal cells.

A more preferred embodiment of the present invention provides that the host cell is a yeast cell.

An even more preferred embodiment of the present invention provides that the yeast strains are selected from Kluyveromyces sp., Pichia sp., Saccharomyces sp., Trichoderma sp. Tetrahymena sp., Yarrowia sp., Hansenula sp., Blastobotrys sp., Candida sp., Zygosaccharomyces sp., and Debaryomyces sp.

Yet another important embodiment of the present invention provides a food composition comprising a fusion protein having the amino acid sequence as defined in SEQ ID No. 1, wherein the food composition is selected from the group consisting of cheese and processed cheese products, yogurt and fermented dairy products, directly acidified counterparts of fermented dairy products, cottage cheese dressing, frozen dairy products, frozen desserts, desserts, baked goods, toppings, icings, fillings, low-fat spreads, dairy-based dry mixes, soups, sauces, salad dressing, geriatric nutrition, creams and creamers, analog dairy products, follow-up formula, baby formula, infant formula, milk, dairy beverages, acid dairy drinks, dairy substitutes, smoothies, milk tea, butter, margarine, butter alternatives, growing up milks, low-lactose products and beverages, medical and clinical nutrition products, protein/nutrition bar applications, sports beverages, confections, meat products, analog meat products, meal replacement beverages, weight management food and beverages, cultured buttermilk, sour cream, yogurt, skyr, leben, lassi, kefir, powder containing a milk protein, and low-lactose products.

### Advantages:

The present invention demonstrates the following advantages:
animal friendly,
reduces greenhouse gas emissions,
cost effective,
increased yield of the proteins
more nutritional value (up to 99% pure protein)
free of lactose, antibiotic, saturated fat, cholesterol, pathogen etc
Alpha casein is used to create micelle formation to formulate the dairy products.
The fusion milk protein can be also using as active carrier to deliver biological agents, has application in paints and rubber industry.
The fusion milk protein has transparency, biodegradability and good technical properties (barrier properties for a polar gas such as O2 and CO2) make casein films innovative materials for packaging.

Without limiting the scope of the present invention as described above in any way, the present invention has been further explained through the examples provided below.

### Experimental Data

### Example 1: Identification and Codon Optimization of the Gene Sequences in Bos indicus

High quality milk producing cow such as Gir cattle (Bos indicus) was identified using NCBI database to identify the specific amino acid sequence and/or nucleotide sequence responsible for targeted protein. Thereafter, further genetic analysis of the identified Gir cattle to identify the specific genetic segment which is responsible for production of desired milk proteins was performed.

In-silico data analysis and codon optimization of identified gene sequences was carried out to obtain the recombinant polynucleotide sequence encoding Alpha-S1-casein and Alpha-S2-casein subunits to further use in the preparation of the fusion milk proteins. Codon optimization of nucleotide sequence is done using GeneScript/IDT software/Snapgene.

### Example 2: Preparation of the Recombinant Plasmid

The inventors of the present invention have further designed the expression vector and primers for gene cloning procedure. Novel expression vector was prepared by combining the different properties and sites of native expression vectors used for Pichia pastoris. Multi-copy integration of the target genes into the genome of P. pastoris was the most efficient strategy. This vector design helped to achieve multiple integration and the inventors have performed modification for antibiotic selection marker site for easy selection of positive transformants at higher concentration of antibiotic.

Further, different promoter and secretary peptides sequences were prepared in designing the constructs for expression of codon optimized gene sequences. The recombinant plasmid was obtained by synthesis of gene segments and multiplication of gene segments using PCR techniques. Plasmids were constructed in vitro by digesting (cutting) DNA fragments with restriction enzymes at specific sites (restriction sites) and then ligating (joining) the resulting fragments. The constructed DNA is usually amplified in E. coli to analyze its structure.

The following protocol was developed for preparing the recombinant plasmid:

i. Preparation of the Recombinant Plasmid

Plasmids (obtained from GeneScript) were diluted to 4 μg (4000 ng) to 50 ng/μL concentration by adding sterile nuclease-free water. A working concentration of 5 ng/μL was prepared for bacterial transformation.

ii. Competent Cell Preparation E. coli DH10β

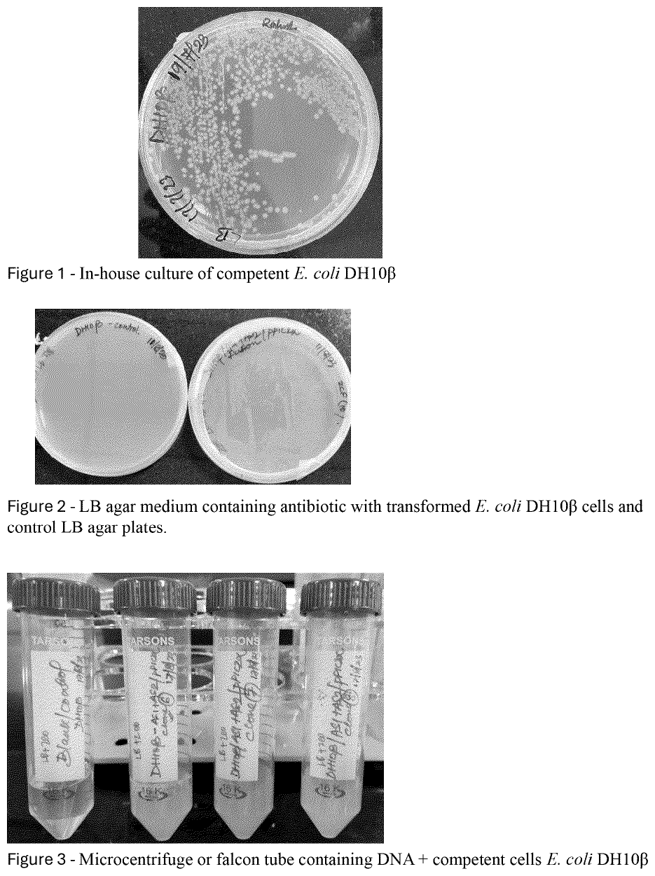

To prepare E. coli DH10β (SLS research private limited) competent cells for propagation of the gene constructs from Gene script cells of E. coli DH10β was inoculated overnight in 5 mL LB broth (purchased from HI Media catalogue no. M1245-500G) medium from glycerol stock (FIG. 1). Recording the optical density (OD) of the culture to ascertain the growth of density of the cells in the inoculum by comparing the with a standard OD reading of 0.05 obtained by setting up a secondary culture in 50 mL LB medium. Once the secondary culture reached OD 0.7-0.8, the culture was immediately incubated at 4° C. for 10 min and cells were then harvested for further steps.

The following subsequent steps were carried out in ice-cooled condition:
The harvested cells were then washed with 0.1 M CaCl₂) (purchased from Sigma catalogue no. C3306-100G) twice and pelleted. To the pellet, 1 mL of solution containing 80 mM CaCl₂) and 20 mM MgCl₂ were added along with 700 μL of 50% Glycerol (purchased from SRL catalogue no. 62417) and mixed thoroughly. Finally, the cell suspension (50 μL) was further aliquoted into each of the sterile tubes for future utility.
   iii. Transformation of AS1 & AS2-pD912 into DH10β Cells (Heat Shock Method)

The competent cells stored in −80° C. were taken and thawed on ice. Agar plates containing the appropriate antibiotic were procured from storage at 4° C. and warmed up to room temperature and then optionally incubated at 37° C. incubator.

1-5 μl of DNA, usually 10 μg-100 ng, were mixed into 20-50 μL of competent cells in a microcentrifuge or falcon tube. Gently mixing by flicking the bottom of the tube with your finger a few times. Incubating the competent cell/DNA mixture on ice for 20-30 mins.

Further, heat shock treatment was carried out for each transformation tube by placing the bottom ½ to ⅔ of the tube into a 42° C. water bath for 30-60 secs preferably 45 seconds, depending on the competent cells. Placing the tubes back on ice for more 2 min. 250-1,000 μl LB or SOC media (without antibiotic) were added to the bacteria and grow in 37° C. shaking incubator for 45 min. All the transformants were plated onto a 10 cm low salt LB agar plate containing the antibiotic zeocin 25 ug. Incubating the plates at 37° C. overnight (FIG. 2, 3).

### Example 3: Transfecting the Recombinant Plasmid to Host Cells

Recombinant plasmid was developed to express a fusion protein containing Alpha-S1 casein and Alpha-S2 casein. Further, this fusion protein also contained a fusion partner (tag) for better expression and purification (SEQ ID No. 7 and 8).

i. Linearization of the Recombinant Plasmid

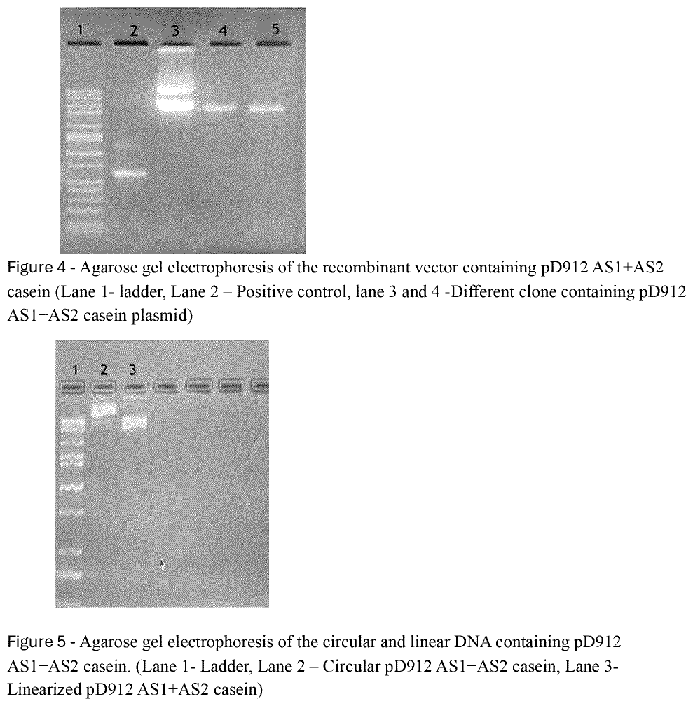

Digesting 20 μg of recombinant plasmid obtained in example 2 in a 200 μl reaction volume according to NEBs guidelines. Similarly, digesting an appropriate control vector. 5 μl of reaction mix was removed before adding the restriction enzyme to act as an un-digested control. The temperature and duration of digestion was as per the recommendation by NEB. 5 μl of undigested control versus 5 μl of digested reaction mix were checked by agarose gel electrophoresis to determine whether the vector was completely linearized, heat-inactivate according to NEBs recommendations (FIG. 4, 5). 1/10 volume of 3M sodium acetate and 2.5 volumes of 100% ethanol were centrifuged to obtain an ethanol precipitate. The pellet DNA were washed with 70% ethanol, air dry, and suspend pellet in 20 μl of deionized sterile water or 10 mM Tris-Cl, pH 8.0. The eluted DNA were measured to check the concentration and/or check quality and yield by agarose gel electrophoresis.

ii. Competent Cell Preparation and Transformation

To prepare Pichia pastoris competent cells and transform them for protein expression. The transformation of recombinant plasmid into Pichia pastoris was carried out by electroporation. Further, screening of positive transformants using PCR and nucleotide sequencing was carried out.

50 mL culture of Pichia pastoris (Atum Bioscience, USA) was grown in Yeast extract, Peptone and Dextrose (YPD) at 30° C. with shaking to an OD600 of 0.8 to 1.0. The cells were harvested and washed with 25 mL of sterile water and centrifuged at 1,500 g for 10 minutes at room temperature. The water was decanted, and the cells were suspended in 1 mL of 100 mM Lithium Chloride (LiCl). The cell suspension was transferred to a 1.5 mL microcentrifuge tube. The cells were pelleted at maximum speed for 15 seconds and the LiCl was removed with a pipet. The cells were resuspended in 400 μL of 100 mM LiCl. 50 μL of the cell suspension was dispensed into a 1.5 mL microcentrifuge tube for each transformation was used immediately.

1 mL sample of single-stranded DNA was boiled for five minutes and then quickly chilled in ice water. For each transformation sample, the following reagents were added in the following order given to the cells:
240 μL 50% PEG
36 μL 1 M LiCl
25 μL1 2 mg/mL single-stranded DNA
Plasmid DNA (5-10 μg) in 50 μL sterile water

The tubes were incubated at 30° C. for 45 minutes without shaking and then heat shocked in a water bath at 42° C. for 40 minutes. To this YPD was added and made up to 1 ml and kept for overnight recovery. 100 μL was plated on YPD plates. The plates were incubated at 2-4 days at 30° C.

### Example 4: Screening and Expression of Fusion Proteins to Obtain Best Transformant

Multiple clones from each plasmid/host were isolated and screened for expression of secreted target fusion proteins. The best clones were regrown in shake flasks to confirm expression and estimate expression levels by SDS-PAGE. The expression of target fusion protein confirmed by Western blot and HPLC methods.

Best strains identified were further evaluated in fermenter for the expression of the target fusion proteins. One or several best transformants were evaluated in lab scale fermenters operated as a set of multiple tanks. Samples were taken during fermentation and analysed for the target proteins by SDS-PAGE. The total broth was harvested by centrifugation.

Thus, after obtaining the best strains after centrifuging the broth, the inventors performed downstream process to isolate and purify the target fusion protein.

i. PTVA Studies to Identify Clone with High Copy Number of Gene for Optimal Expression of Targeted Milk Proteins

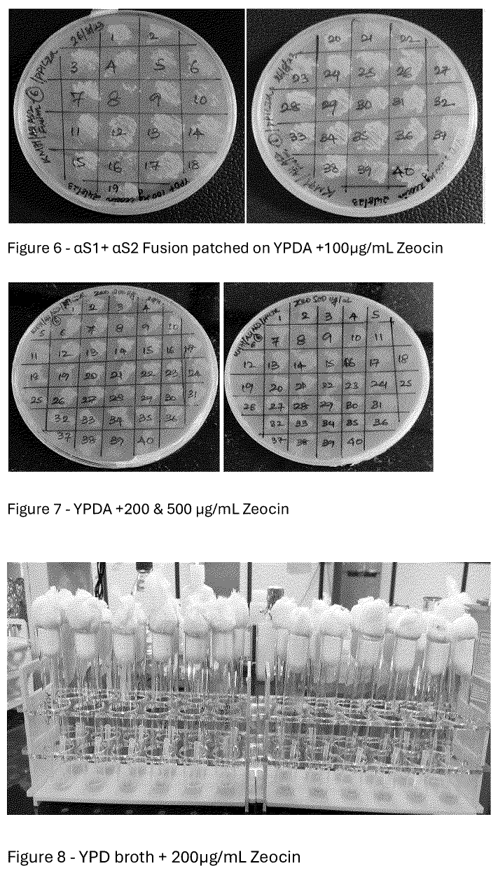

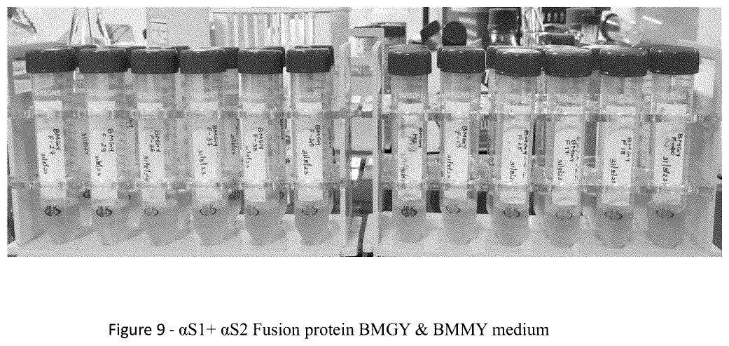

The recombinant clones obtained in example 3 were grown on a higher concentration of zeocin to create multi-copy clones with a range of copy no. to systematically evaluate the effects of strain engineering efforts also called post-transformational vector amplification (PTVA). FIG. 6 describes stepwise increase in antibiotic concentration results in strains with a higher copy number of the gene of interest, finally resulting in higher titres of protein expression. PTVA uses a single vector for transformation (containing Zeocin™ resistance marker).

Selection is originally on a low concentration of the corresponding antibiotic, but the cells are increasingly subjected to higher concentrations. Only colonies that have multiple copies of the resistance gene (and therefore multiple copies of the heterologous gene) will be able to survive on the highest concentrations. Jackpot colonies are reported in 6% of all clones tested.

In PTVA, instead of direct selection on high concentrations of antibiotics, cells are spotted onto agar plates with increasing antibiotic concentrations with approximately 5 days growth in between each step. The results determine that the particular clone grows up to 1000 ug/ml concentration (FIG. 6).

ii. Screening Ad Expression of Fusion Proteins

Yeast extract peptone dextrose (YEPD) is a nutrient-rich and complex broth used for general growth and storage. Buffered Glycerol-complex Medium (BMGY) is used in protein expression studies to control the pH of the medium, decrease protease activity, and generate biomass. Buffered Methanol-complex Medium (BMMY) is used in protein expression studies to control the pH of the medium, decrease protease activity, and induce protein expression. The inventors initially inoculated clones in YPD which is a nutrient-rich medium which ensures a healthy population of cells is taken forward for protein production.

### Compositions of the Media Used:

| YPD | BMGY (1L) | BMMY (1L) |
|---|---|---|
| 1% Yeast extract | 1% yeast extract | 1% yeast extract |
| 2% Peptone | 2% peptone | 2% peptone |
| 2% Dextrose | 100 mM potassium | 100 mM potassium |
| | phosphate, pH 6.0 | phosphate, pH 6.0 |
| | 1.34% YNB | 1.34% YNB |
| | 4 × 10⁻⁵% biotin | 4 × 10⁻⁵% biotin |
| | 1% glycerol | 0.5% methanol |

After overnight growth, the cells are transferred to BMGY and grown for 48 hours (See FIG. 7) till the OD600 reaches approximately 10. Growing the cells in BMGY helps in attaining high cell densities before starting methanol induction which in turn can lead to higher protein production. Then the cells are transferred to BMMY and grown for 144 hrs. The induction is done with 0.5% methanol.

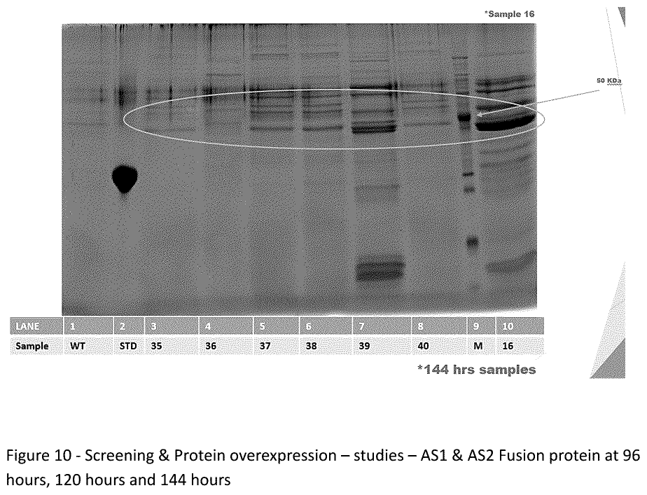

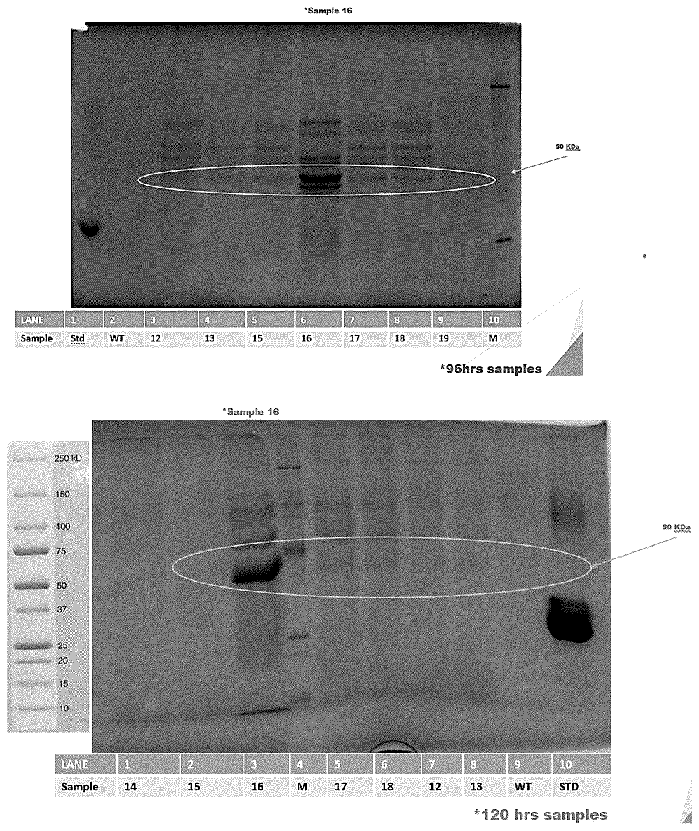

FIG. 10 (a, b, c) shows the SDS page results for protein overexpression studies AS1 & AS2 Fusion protein (sample 16) at 96 hours, 144 hours and 120 hours. These results demonstrate the sample 16 is the most ideal to perform further purification studies by the band observed around 50 kDa.

iii. Detection of Target Fusion Protein in Crude Transformed Host Cells by Dot Blot Assay

The inventors employed dot blot assay for detecting, analysing, and identifying proteins, similar to the western blot technique but differing in that protein samples are not separated electrophoretically but are spotted through circular templates directly onto the membrane (PVDF or Nitrocellulose membrane). The concentration of proteins in crude preparations (such as culture supernatant) can be estimated semi-quantitatively by using specific antibody against the purified fusion protein. Through Dot Blot assay, the inventors detected the protein of interest expression in diverse samples, from P. pastoris crude extracts. The adsorption of crude extracts on nitrocellulose membranes allows easy testing of hundreds of samples; this method also validates the evaluation of relative expression levels of proteins. This procedure is also suitable for other applications such as the optimization of protein expression conditions as well as the monitoring of the jackpot clones.

### Dot Blot Method:

### Reagent Preparation:

### 10×TBS (Tris Buffered Saline) Buffer:

| Component | Amount |
|---|---|
| Tris base | 12.114 |
| NaCl | 43.83 |
| water | Up to 500 ml |
| pH | 7.4 |

Dissolving tris base and NaCl in 800 ml distilled water, adjust the pH 7.4 and make the volume up to 500 ml. To prepare 500 ml of 1×TBS buffer, the inventors added 50 ml of 10×TBS in 450 ml of distilled water.

Blocking Buffer (1% BSA with 0.5% Tween 20):

| Component | Amount |
|---|---|
| BSA | 1 gm |
| Tween 20 | 0.5 ml |
| 1× TBS | Up to 100 ml |

### Protocol:

Activation of pvdf membrane was carried out by immersing the membrane in 100% methanol for 15 seconds. Washing the membrane with distilled water for 2 minutes followed by equilibrating the membrane in 1×TBS buffer for 5 minutes.

### For Nitrocellulose Membrane:

The inventors used the membrane pvdf washed with distilled water onto which 2-5 μl from sample 16 was pipetted onto the membrane and the membrane was allowed to dry. After drying of membrane, the membrane was incubated in blocking buffer for 1 hour at room temperature in shaking condition. The blocking buffer was decanted, and the primary antibody solution diluted in blocking buffer was added; the primary antibody was diluted according to antibody manual. The membrane was incubated for 3-4 hour with shaking at room temperature, which may extend to overnight incubation. The primary antibody solution was removed, and the membrane was washed with TBS buffer three times 5 minutes each.

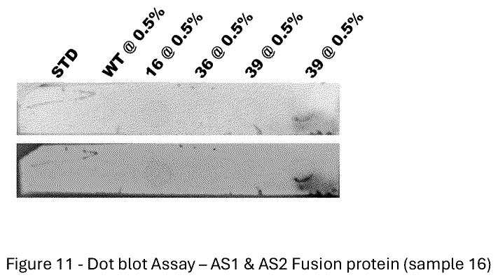

The membrane was then incubated with secondary antibody solution diluted in blocking buffer for 1 hour with shaking at room temperature; the primary antibody was diluted according to the antibody manual. The secondary antibody solution was removed, and the membrane was washed with TBS buffer three times 5 minutes each. The membrane in substrate solution was incubated until spots are visible. FIG. 11 shows the results of the dot blot assay showing colour development and positive for protein expression for sample 16.

iv. Isolation of Target Fusion Protein from the Sample 16

The inventor used affinity chromatography techniques using the HIS tag present on the target protein using Nickle nitriloacetic acid resin. His-tagged protein purification requires the His-tag and Ni-NTA interaction, which is based on the selectivity and high affinity of Ni-NTA (nickel nitrilotriacetic acid) resin for proteins containing an affinity tag of, e.g., six consecutive Histidine residues. NTA, which has four chelating sites for nickel ions, binds nickel more tightly than metal-chelating purification systems like IDA (iminodiacetic acid), which have only three sites available for interaction with metal ions. The his-tag has a high affinity for these metal ions and binds strongly to the IMAC column. Most other proteins in the lysate will not bind to the resin or bind only weakly.

### Procedure:—

### Resin Washing and Packing:

The resins were shipped and stored in a 50% suspension of 20% ethanol. Prior to first usage, the Ni-NTA resin was washed with metal-free water more than 10 CV. The resin was settled at least three times using 5 CV of metal-free water to remove the small broken particles or debris. (If sanitization is necessary, soak the resin in 5 CV of 0.5M NaOH for more than 1 hour.) The resin is filtered and washed with water to a pH of 7 before packing. 0.45 um nitrocellulose membrane was cut with the size equivalent to the internal diameter of the syringe and place it inside the bottom of the syringe so that while packing, the resin won't leak out of the syringe. Once the resin is settled at the bottom, the water is carefully removed from the top of the resin so that the bed of resin won't be disturbed.

Column equilibration:—Once the resin is packed, the column is equilibrated with 50 mM of sodium phosphate buffer at pH 7 for 2 CV.

Sample preparation and loading: The pH of the sample is adjusted to equivalent to the equilibration buffer using any weak acid or base, as after the equilibration, the resin should not get a pH shock from sample loading. After pH adjustment, the sample is passed through the 0.2 um syringe filter and loaded into the syringe carefully without disturbing the bed, and the volume of the load should not be less than 5CV. The flowthrough of the syringe is collected for further analysis.

Column washing:—Once the flowthrough is collected for sample loading, the column is washed using a washing buffer that contains 150 mM NaCl and 10 mM imidazole in 50 mM sodium phosphate buffer of pH 7 four times. The column wash is given with 2CV of the washing buffer to remove the nonspecific binding of molecules with the resin. The flowthrough of the syringe is collected for further analysis.

Column Elution:—Once the flowthrough is collected from the column washing, the step-elution of the column is initiate which is carried out in a 4-step elution, and the following buffer will be used:
E1—50 mM imidazole and 150 mM NaCl in 50 mM sodium phosphate at pH 7 (0.5 CV)
E2—100 mM imidazole and 150 mM NaCl in 50 mM sodium phosphate at pH 7 (0.5 CV)
E3—250 mM imidazole and 150 mM NaCl in 50 mM sodium phosphate at pH 7 (0.5 CV)
E4—500 mM imidazole and 150 mM NaCl in 50 mM sodium phosphate at pH 7 (0.5 CV)

Collect the flowthrough of each elution separately for further analysis.

Resin Regeneration:—In case some proteins are deposited on the resins, denaturing chemicals such as urea and organic solvents were used to clean the resins. To remove the endotoxins and HCP, the resins were washed with 0.5M or 1 M NaOH for an extended period of time, then equilibrated with the appropriate binding buffer for 20 CV. The column is washed with 50 mM sodium phosphate buffer pH 7 for 10 CV before the next use. For extended storage, it is recommended that the column and resin be stored in 0.02% sodium azide or 20% ethanol at 2-8° C.

The affinity chromatography yielded around 0.5 mg/ml of the target fusion protein which was further used for HPLC analysis.

v. Purification and Estimation of Protein Using HPLC Method:

### Materials and Methods:

Chemicals/Reagents: Trifluoroacetic acid (TFA), Acetonitrile (ACN), Mili Q Water

### Mobile Phase Trail:

Solution A—(Mili Q+0.1% TFA) and Solution B—(ACN+0.1% TFA)
Solution A—(Mili Q+0.1% TFA) and Solution B—(ACN:Mili Q:TFA) (90:10:0.1%) % v/v
Stationary Phase: ZORBAX 300SB-C18 (4.6×250 mm, 5—micron)
Sample Preparation: Diluent—1M Phosphate buffer saline (PBS). Weigh 10 mg of α-Casien pure standard and transfer into 2 ml of Eppendorf tube, add 1 ml of PBS.

### Chromatographic Conditions:

Mobile Phase—Sol A—(Mili Q+0.1% TFA) and Sol B—(ACN:Mili Q:TFA) (90:10:0.1%) % v/v
Stationary phase—ZORBAX 300SB-C18 (4.6×250 mm, 5—micron)
Temperature—40° C.
Flow rate—1 ml/min
Detection—215 nm
Injection Volume—20 μl
Total Run Time—40 min

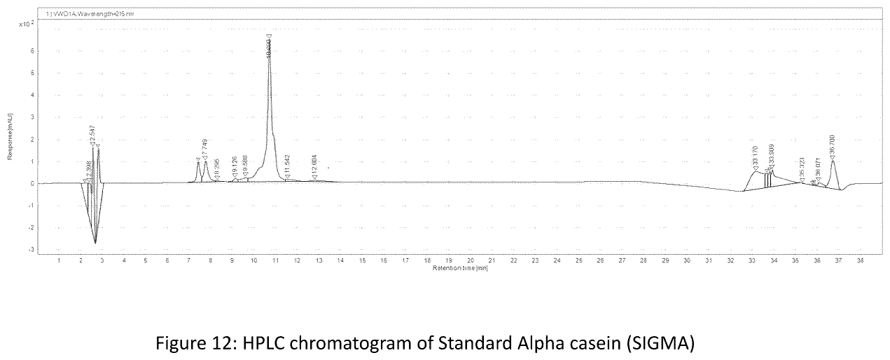

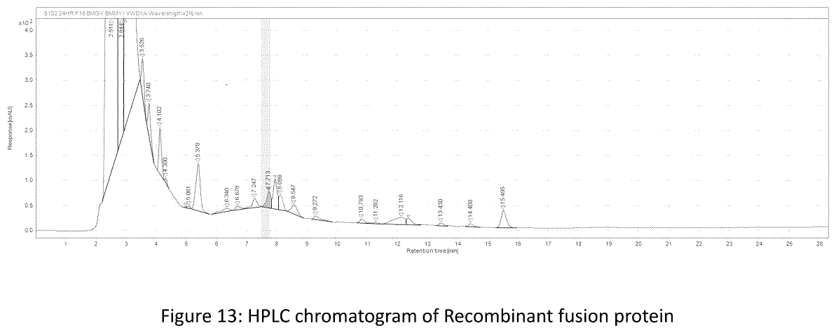

FIGS. 12 and 13 disclose the results HPLC chromatogram of Standard Alpha casein (SIGMA) and recombinant fusion protein which demonstrates that the optimized protocol for HPLC using different solvent, temperature and column results in a detection of 0.5 mg/ml yield of target fusion protein using standard alpha casein Peak in HPLC analysis. This method helps for accurate quantification of targeted protein.

The foregoing broadly outlines the features and technical advantages of the present disclosure in order that the detailed description of the disclosure that follows may be better understood. It should be appreciated by those skilled in the art that the conception and specific embodiment disclosed may be readily utilized as a basis for modifying the disclosed methods or for carrying out the same purposes of the present disclosure.

It must be noted that as used herein, the singular forms “a”, “an”, and “the”, include plural references unless the context clearly indicates otherwise. Thus, for example, reference to “an expression cassette” includes one or more of the expression cassettes disclosed herein and reference to “the method” includes reference to equivalent steps and methods known to those of ordinary skill in the art that could be modified or substituted for the methods described herein.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well-known in the art. Generally, nomenclatures used in connection with techniques of biochemistry, enzymology, molecular and cellular biology, microbiology, genetics and protein and nucleic acid chemistry and hybridization described herein are those well-known and commonly used in the art.

The methods and techniques of the present invention are generally performed according to conventional methods well-known in the art and as described in various general and more specific references that are cited and discussed throughout the present specification unless otherwise indicated. See, e. g., Sambrook et al., Molecular Cloning: A Laboratory Manual, 3rd ed., Cold Spring Harbor Laboratory Press, Cold Spring Harbor, N. Y. (2001); Ausubel et al., Current Protocols in Molecular Biology, J, Greene Publishing Associates (1992, and Supplements to 2002); Handbook of Biochemistry: Section A Proteins, Vol 11976 CRC Press; Handbook of Biochemistry: Section A Proteins, Vol 111976 CRC Press. The nomenclatures used in connection with, and the laboratory procedures and techniques of, molecular and cellular biology, protein biochemistry, enzymology and medicinal and pharmaceutical chemistry described herein are those well-known and commonly used in the art.

We claim:

1. A fusion protein encoded by a recombinant polypeptide sequence having an amino acid sequence having 90% similarity to SEQ ID No. 1, wherein the said fusion protein is an animal free milk protein that mimic the naturally occurring milk proteins.

2. The fusion protein as claimed in claim 1, wherein the recombinant polypeptide sequence, SEQ ID No. 1, comprises an amino acid sequence having 90% similarity to SEQ ID No. 3 and an amino acid sequence having 90% similarity to SEQ ID No. 5.

3. The fusion protein as claimed in claim 1, wherein the fusion protein bovine milk proteins are selected from any bovine milk proteins selected from the group comprising of Beta-casein, Kappa-casein, Alpha-S1-casein, Alpha-S2-casein, Alpha-lactalbumin, Beta-lactoglobulin, Lactoferrin, and/or Transferrin.

4. The animal free fusion protein as claimed in claim 1, wherein the bovine milk proteins are Alpha-S1-casein and Alpha-S2-casein.

5. The bovine milk protein as claimed in claim 4, wherein the amino acid sequences encoding Alpha-S1-casein and Alpha-S2-casein have been obtained from Gir cattle (Bos indicus).

6. A process for the production of animal-free milk proteins in recombinant host cells, the process comprising the steps of:
i. identifying high quality milk producing Gir cattle (Bos indicus) by using various bioinformatics techniques and analysis;
ii. performing genetic analysis of identified Gir cattle from step (i) to identify the specific genetic segment which is responsible for production of desired milk proteins;
iii. performing in-silico data analysis and codon optimization of gene sequences for the requisite milk proteins to obtain the gene sequences;
iv. Preparing the recombinant vector comprising the recombinant polynucleotide sequence having SEQ ID No. 2 obtained from step (iii);
v. transforming the recombinant vector obtained in step (iv) into the host cell; and
vi. culturing the recombinant host cells to express the animal free milk protein.

7. The process as claimed in claim 6, wherein the step (iv) of the method comprises the steps of:
a) preparing DNA fragment(s) from a linearized plasmid vector by restriction enzyme digestion or by PCR techniques;
b) amplifying DNA fragment(s) obtained in step (a) from the insert by PCR techniques while ensuring that the sequence at the end of the fragment is homologous with that of the plasmid;
c) mixing amplified DNA obtained in step (b) with competent cells in a microcentrifuge or falcon tube, followed by gentle mixing by flicking the bottom of the tube a few time;
d) incubating the competent cell/DNA mixture obtained in step (c) on ice;
e) performing heat shock transformation by placing the microcentrifuge tube into a water bath followed by placing the tubes back on ice for to obtained the transformed E. coli cells;
f) adding Luria Broth (LB) or (SOC) media to the tube containing the transformed E. coli cells and incubating the tubes in a shaking incubator;
g) plating the grown transformed cells onto a low salt LB agar plate containing the antibiotic and incubating the plates overnight;
h) selecting the positive transformants by identifying the plasmid vector marker;
i) recovering the constructed vector from the transformed host cell into a second host cell to amplify and identify the structure of the plasmid with restriction enzyme digestion and DNA sequencing.

8. The process as claimed in claim 6, wherein the step (v) of the method comprises the steps of:
a) digesting the recombinant vector obtained in step (iv) of the process as claimed in claim 6, with the restriction enzyme at an appropriate temperature and time to obtain the linear vector;
b) boiling the single-stranded linear vector followed by immediate chilling in ice water;
c) culturing host cells in YPD media with shaking followed by harvesting the cells and washing with sterile water;
d) centrifuging the solution obtained in step (c) at room temperature and the water from the solution was decanted followed with suspending the cells in LiCl;
e) transferring the cell suspension obtained in step (d) to a microcentrifuge tube and centrifuging the tube at maximum speed followed by removing the LiCl with a pipette;
f) resuspending the cells in LiCl followed by dispensing the cell suspension into a microcentrifuge tube for carrying out transformation studies;
g) adding to the transformation tubes PEG, LiCl, single-stranded plasmid DNA obtained in step (f) followed by incubating the tube without shaking;
h) subjecting the tubes to heat shock by placing them in a water bath;
i) adding YPD to the tubes obtained in step (h) and kept for overnight for culturing the transformed cells followed by plating the cells on YPD plates and incubating the plates to obtain the transformed cells.

9. The process as claimed in claim 6, wherein the step (vi) of the method comprises the steps of:
a) screening of positive transformant host cells obtained in step (v) of the method as claimed in claim 6, using PCR and nucleotide sequencing;
b) isolating and screening multiple clones from each transformed host cells for expression of secreted target proteins;
c) regrowing the best clones isolated in step (ii) in shake flasks to confirm expression;
d) extracting target fusion protein from the transformed host cells by performing affinity chromatography to isolate the target fusion proteins;
e) performing downstream processing to purify the target fusion milk proteins.

10. The method as claimed in claim 6, wherein the host cell is selected from bacterial, yeast or fungal cells.

11. The method as claimed in claim 6, wherein the host cell is a yeast cell.

12. The method as claimed in claim 9, wherein the downstream purification process in step (e) can be carried out by any method including centrifugation, column chromatography and/or membrane filtration.

13. A composition comprising the fusion protein as claimed in claim 1 along with pharmaceutically or nutraceutically acceptable carriers or vehicles.

14. A nucleic acid sequence encoding the recombinant fusion protein encoded by the recombinant polynucleotide sequence having 90% similarity to SEQ ID No. 2

15. The nucleic acid as claimed in claim 14, wherein the recombinant polynucleotide sequence, SEQ ID No. 2, comprises polynucleotide sequences having 90% similarity to SEQ ID No. 4 and SEQ ID No. 6.

16. A recombinant vector comprising the nucleic acid sequence as claimed in claim 14, wherein the nucleic acid sequence encoding the recombinant fusion protein encoded by the recombinant polynucleotide sequence having 90% similarity to SEQ ID No. 2.

17. The recombinant vector as claimed in claim 16, wherein the polynucleotide sequence operably linked to a promoter, a nucleotide sequence encoding a ‘HIS’ tag, a nucleotide sequence encoding a bacterial resistance marker and a transcription terminator.

18. A recombinant host cell comprising the nucleic acid sequence encoding the recombinant fusion protein having 90% similarity to SEQ ID No. 1 as claimed in claim 1.

19. The host cell as claimed in claim 18, wherein the host cell is selected from bacterial, plant, yeast or fungal cells.

20. The host cell as claimed in claim 18, wherein the host cell is a yeast cell.

21. A food composition comprising a fusion protein having the amino acid sequence having 90% similarity to SEQ ID No. 1 as claimed in claim 1, wherein the food composition is selected from the group consisting of cheese and processed cheese products, yogurt and fermented dairy products, directly acidified counterparts of fermented dairy products, cottage cheese dressing, frozen dairy products, frozen desserts, desserts, baked goods, toppings, icings, fillings, low-fat spreads, dairy-based dry mixes, soups, sauces, salad dressing, geriatric nutrition, creams and creamers, analog dairy products, follow-up formula, baby formula, infant formula, milk, dairy beverages, acid dairy drinks, dairy substitutes, smoothies, milk tea, butter, margarine, butter alternatives, growing up milks, low-lactose products and beverages, medical and clinical nutrition products, protein/nutrition bar applications, sports beverages, confections, meat products, analog meat products, meal replacement beverages, weight management food and beverages, cultured buttermilk, sour cream, yogurt, skyr, leben, lassi, kefir, powder containing a milk protein, and low-lactose products.


SEQUENCE LISTING (6 sequences)

SEQ ID NO: 1 - protein, 426 aa, synthetic construct
RPKHPIKHQGLPQEVLNENLLRFFVAPFPEVFGKEKVNELSKDIGSESTEDQAMEDIKQMEAESISSSEEIVPNSVEQKHIQKXDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLWENLYFQSKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYLENLYFQSHHHHHH

SEQ ID NO: 2 - DNA, 1281 bp, synthetic construct
  source 1..1281: mol_type = other DNA
aggcccaaacaccctatcaaacaccaaggattgccacaagaagtgttaaatgagaatttattaagatttttcgtagctcctttccctgaggtcttcggtaaagaaaaagtgaatgagctatccaaagatattggatctgagtcaacagaggaccaagctatggaggatatcaaacagatggaagctgaatccatatccagttccgaggagatcgtaccaaactctgtcgaacaaaagcacatccagaaagacgatgttcctagtgaaagatacctgggttacctagaacaactgttaagattgaagaaatataaagtgccacaattggaaattgtccccaactctgctgaagaaagactacattctatgaaagagggcatacatgctcagcaaaaagagccaatgattggcgtcaaccaggagttggcatacttctatcccgagttgttcagacaattttaccagttggatgcttatccaagtggtgcttggtactatgtccccctgggcacccagtacacagatgcacctagtttctctgatataccaaaccctattggttcagaaaactccggcaaaacgacaatgcccttatgggagaacctgtacttccaatctaaaaatactatggagcacgtgtcatcctcagaagaatccataatatcccaggagacatataaacaagaaaaaaatatggcaattaatccaagtaaagagaacctatgttctacattctgcaaggaagttgttagaaatgccaatgaggaagagtactcaataggttcctcctcagaggaaagtgccgaggttgccacagaagaagtaaaaattactgttgacgataaacattaccagaaggctctaaacgagatcaatcaattctatcaaaaattcccccagtatctacaatatctttaccaaggtcccatagtactgaacccttgggatcaagttaaaaggaatgctgttcctatcacgcctacattaaatagggagcaattatccacctccgaagagaactcaaagaagaccgtggacatggaatcaacagaagtgttcacgaagaaaacgaaattaacagaggaggagaagaatagattaaattttttgaaaaaaatctcacaacgttatcagaaattcgccttgccccagtacttaaagaccgtctatcagcatcaaaaggccatgaagccctggatccaacccaagacaaaagtgattccctatgtcaggtacttagagaacctgtacttccaatctcaccatcatcaccaccactga

SEQ ID NO: 3 - protein, 199 aa, Bos indicus
RPKHPIKHQGLPQEVLNENLLRFFVAPFPEVFGKEKVNELSKDIGSESTEDQAMEDIKQMEAESISSSEEIVPNSVEQKHIQKDDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 4 - DNA, 639 bp, Bos indicus
  source 1..639: mol_type = genomic DNA
aggcccaaacaccctatcaaacaccaaggattgccacaagaagtgttaaatgagaatttattaagatttttcgtagctcctttccctgaggtcttcggtaaagaaaaagtgaatgagctatccaaagatattggatctgagtcaacagaggaccaagctatggaggatatcaaacagatggaagctgaatccatatccagttccgaggagatcgtaccaaactctgtcgaacaaaagcacatccagaaagacgatgttcctagtgaaagatacctgggttacctagaacaactgttaagattgaagaaatataaagtgccacaattggaaattgtccccaactctgctgaagaaagactacattctatgaaagagggcatacatgctcagcaaaaagagccaatgattggcgtcaaccaggagttggcatacttctatcccgagttgttcagacaattttaccagttggatgcttatccaagtggtgcttggtactatgtccccctgggcacccagtacacagatgcacctagtttctctgatataccaaaccctattggttcagaaaactccggcaaaacgacaatgcccttatgggagaacctgtacttccaatctcaccatcatcaccaccactga

SEQ ID NO: 5 - protein, 207 aa, Bos indicus
KNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 6 - DNA, 663 bp, Bos indicus
  source 1..663: mol_type = genomic DNA
aaaaatactatggagcacgtgtcatcctcagaagaatccataatatcccaggagacatataaacaagaaaaaaatatggcaattaatccaagtaaagagaacctatgttctacattctgcaaggaagttgttagaaatgccaatgaggaagagtactcaataggttcctcctcagaggaaagtgccgaggttgccacagaagaagtaaaaattactgttgacgataaacattaccagaaggctctaaacgagatcaatcaattctatcaaaaattcccccagtatctacaatatctttaccaaggtcccatagtactgaacccttgggatcaagttaaaaggaatgctgttcctatcacgcctacattaaatagggagcaattatccacctccgaagagaactcaaagaagaccgtggacatggaatcaacagaagtgttcacgaagaaaacgaaattaacagaggaggagaagaatagattaaattttttgaaaaaaatctcacaacgttatcagaaattcgccttgccccagtacttaaagaccgtctatcagcatcaaaaggccatgaagccctggatccaacccaagacaaaagtgattccctatgtcaggtacttagagaacctgtacttccaatctcaccatcatcaccaccactga